(19) United States
(12) Patent Application Publication
(10) Pub. No.: US 2023/0113613 A1
(43) Pub. Date: Apr. 13, 2023

(54) Virtual Tasting Systems and Methods

(71) Applicant: Aka Foods LTD, Tel Aviv (IL)

(72) Inventors: Alex Bronstein, Haifa (IL); Ariel Harpaz, Mevaseret Zion (IL); David H. Silver, Haifa (IL); Tal Knafo, Haifa (IL)

(21) Appl. No.: 17/950,634

(22) Filed: Sep. 22, 2022

Related U.S. Application Data
(63) Continuation of application No. 17/691,662, filed on Mar. 10, 2022.
(60) Provisional application No. 63/253,214, filed on Oct. 7, 2021.

(51) Int. Cl. G01N 33/02; G06F 30/20; A47J 43/07

(52) U.S. Cl. CPC G01N 33/02; G06F 30/20; A47J 43/07

(57) ABSTRACT

Example virtual tasting systems and methods are described. In one implementation, a virtual tasting system identifies multiple food products, multiple tasters, and multiple questions. The virtual tasting system then identifies a full set of quadruplets of the form: first food product, second food product, taster, question. A subset of quadruplets from the full set of quadruplets are then identified. The virtual tasting system then measures a value associated with each quadruplet from the subset of quadruplets. The virtual tasting system then outputs estimated values associated with every triplet in a full set of triplets of the form: food product, taster, question.

### RELATED APPLICATIONS

This application is a Continuation in Part of U.S. application Ser. No. 17/691,662, entitled “Food Processing Systems and Methods,” filed Mar. 10, 2022, the disclosure of which is incorporated herein by reference in its entirety. This application also claims the priority benefit of U.S. Provisional Application Ser. No. 63/253,214, filed on Oct. 7, 2021, the disclosure of which is incorporated herein by reference in its entirety.

### TECHNICAL FIELD

The present disclosure relates to systems and methods that provide virtual tasting of one or more food products or ingredients.

### BACKGROUND

Existing techniques for creating new food products and associated recipes often require significant experimentation and considerable human tasting. Additionally, these existing techniques may require an experienced chef or other food product designer to create new combinations of ingredients that are likely to taste good to a human.

The techniques that require an experienced chef, significant experimentation, and many human tasting tests can be expensive and time-consuming. Further, those techniques can be limited to the chef's personal experience with different types of recipes and ingredients. The need exists for systems and methods that can create new food products and develop new recipes in a manner that accesses a wider universe of ingredients, is less expensive, and requires less trial-and-error to implement.

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 is a block diagram illustrating an environment within which an example embodiment may be implemented.

FIG. 2 is a flow diagram illustrating an embodiment of a process for preparing and testing new preparation instructions.

FIG. 3 is a block diagram illustrating an embodiment of a process flow for predicting characteristics of preparation instructions.

FIG. 4 is a block diagram illustrating an embodiment of a process flow for optimizing creation of new preparation instructions.

FIG. 5 is a flow diagram illustrating an embodiment of a process for implementing a virtual tasting room and similar environments.

FIG. 6 illustrates an example embodiment of a table containing data related to the systems and methods described herein.

FIG. 7 is a flow diagram illustrating an embodiment of a process for implementing a virtual tasting system.

FIG. 8 illustrates an example block diagram of a computing device.

### DETAILED DESCRIPTION

The perception of taste is a psychological experience based primarily on the structural and chemical molecular properties of various ingredients and their interactions with the taste and smell receptors and themselves.

In some embodiments, the systems and methods discussed herein identify the objective properties of ingredients and molecules. The objective properties of various ingredients and molecules are translated into a subjective tasting experience that may include, for example, its savor, smell, texture, and mouthfeel.

As discussed herein, the described systems and methods can evaluate actual human tasting results as well as the objective properties using a food processing unit (FPU) and other components or systems to generate reliable distributions of predicted user responses from a small number of actual human tastings. Thus, the systems and methods may provide alternate materials or ingredients that can be mixed and prepared to provide tasting experiences similar to traditional foods with minimal human tasting activities.

In some embodiments, the described systems and methods may identify new ingredients and preparation instructions for traditional foods that eliminate animal products, eliminate certain food allergens, replace expensive ingredients, replace ingredients that are in short supply, and the like.

In the following disclosure, reference is made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

An implementation of the devices, systems, and methods disclosed herein may communicate over a computer network. A “network” is defined as one or more data links that enable the transport of electronic data between computer systems and/or modules and/or other electronic devices. When information is transferred or provided over a computer network or another communications connection (either hardwired, wireless, or a combination of hardwired or wireless) to a computer, the computer properly views the connection as a transmission medium. Transmission media can include a computer network and/or data links, which can be used to carry desired program code means in the form of computer-executable instructions or data structures and which can be accessed by a general purpose or special purpose computer. Combinations of the above should also be included within the scope of computer-readable media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter is described in language specific to structural features and/or methodological acts, it is to be understood that the subject matter defined in the appended claims is not necessarily limited to the described features or acts described herein. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a communication network, both perform tasks. In a distributed system environment, program modules may be located in both local and remote memory storage devices.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the description and claims to refer to particular system components. As one skilled in the art will appreciate, components may be referred to by different names. This document does not intend to distinguish between components that differ in name, but not function.

It should be noted that the sensor embodiments discussed herein may comprise computer hardware, software, firmware, or any combination thereof to perform at least a portion of their functions. For example, a sensor may include computer code configured to be executed in one or more processors, and may include hardware logic/electrical circuitry controlled by the computer code. These example devices are provided herein for purposes of illustration, and are not intended to be limiting. Embodiments of the present disclosure may be implemented in further types of devices, as would be known to persons skilled in the relevant art(s).

At least some embodiments of the disclosure are directed to computer program products comprising such logic (e.g., in the form of software) stored on any computer useable medium. Such software, when executed in one or more data processing devices, causes a device to operate as described herein.

Various terms are used in this specification to describe systems, methods, ingredients, molecular structures, processing steps, data, and the like. For example, the following terms are briefly described for a particular embodiment. It should be understood that the following descriptions are presented by way of example only, and not limitation. It will be apparent to persons skilled in the relevant art that different descriptions may be provided for these terms without departing from the spirit and scope of the disclosure.

In some embodiments, vectors are represented as bold, roman, lower case. Scalars may be represented as italics. Matrices may be represented as bold, roman, upper case. T may represent transpose.

The systems and methods define the fundamental and indivisible constituent of a food product as a simple (mono-molecular) ingredient—a substance containing only one type of molecules. Simple ingredients may be mixed in some proportions forming composite ingredients. Ingredients may be further subjected to various types of transformations such as heating or cooling. From this perspective, any food product can be described as a sequence of mixing and transformation operations applied initially to the raw simple ingredients, and to the intermediate products until the final product is obtained. As discussed herein, such a sequence is referred to as “preparation instructions”, while the list of the initial ingredients and their quantities is referred to as the “formula” of the food product.

The discussion herein refers to the set of characteristics of a food product pertaining to the flavor perception it generates as to its “flavor profile”. A flavor profile may contain objective characteristics characterizing the sensory response (for example, the binding affinities of the different molecular constituents of the food product to a set of known taste receptor proteins, or mechanical properties such as elasticity as a function of temperature), as well as subjective characteristics (for example, a verbal description of the food product's taste and smell and its comparison to other reference products in the sense of some fixed flavor features such as sweetness, bitterness, sourness, texture, and mouthfeel).

In some embodiments, an ingredient is a natural or synthetic mixture of molecules in some concentrations (e.g., relative amounts). An ingredient can be simple (mono-molecular) or composite (comprising more than one molecule). Concentrations and the constituent molecules of an ingredient can be determined by chemical analytic methods such as liquid chromatography (LC) and mass spectrometry (MS).

A formula may include a list of ingredients with their quantities, which is different from a chemical formula. A mixture is the result of mixing various ingredients according to a formula. The chemical composition of a mixture may change based on chemical reactions between the constituent molecules.

A transformation is an operation or process applied to an ingredient, such as baking at 180 degrees Celsius for 5 minutes.

A preparation instruction is a directed graph starting from a formula and applying a sequence of mixtures and transformations resulting in a single output food (prepared according to the preparation instructions). In some embodiments, a food may also be an ingredient.

A subjective flavor profile may include a description of how the taste/smell of an ingredient is perceived by a human taster. It may also include one or more keywords that approximate the evoked perception, a vector of scores numerically grading different flavor features (sweetness, bitterness, and the like), or a comparison of the above features to another ingredient (e.g., A is sweeter than B, A is more bitter than C, A is as sour as D, and the like).

An objective flavor profile may include measurable physical and chemical characteristics such as pH, viscosity, hardness, and the like.

A flavor profile may be a combination of the subjective flavor profile and the objective flavor profile.

In some embodiments, the systems and methods described herein may receive an ingredient list and a reference food. Based on the ingredient list and reference food, the systems and methods generate preparation instructions for a particular food item using one or more alternate ingredients than the traditional preparation instructions.

The systems and methods discussed herein are further capable of providing a virtual tasting room for testing new food products, food ingredients, and the like. These systems and methods can predict which users may like a particular food product and which users may be the best testers of new food products (or comparisons between two or more similar food products).

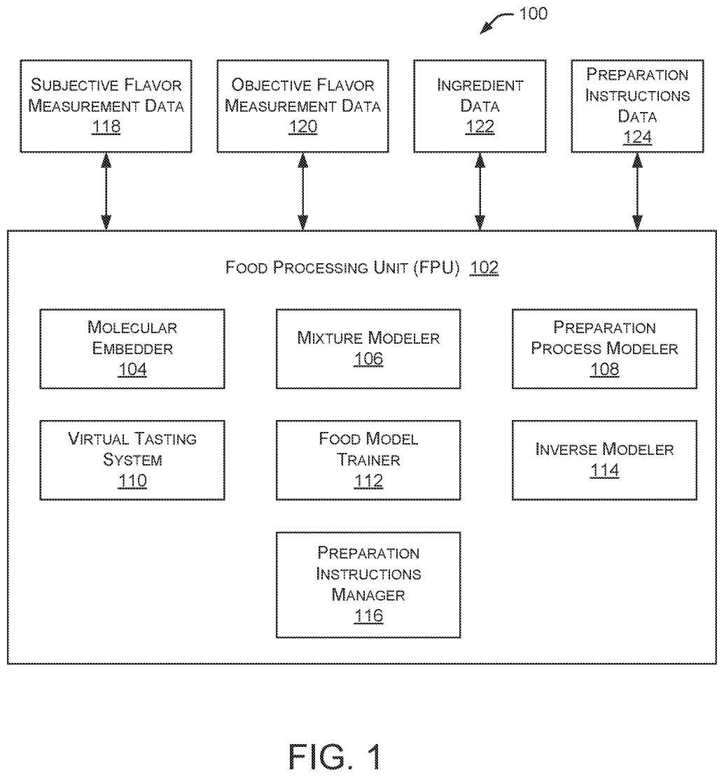

FIG. 1 is a block diagram illustrating an environment 100 within which an example embodiment may be implemented. As shown in FIG. 1, environment 100 includes a food processing unit (FPU) 102 that may implement one or more processes to replicate food characteristics using a mixture of food items, ingredients, and the like. In some embodiments, FPU 102 may replicate food characteristics based on information (e.g., data-driven elements) stored in one or more databases, as discussed herein.

In some implementations, FPU 102 may contain or access a digitization of one or more food features using various combinations of subjective food tastings, mixture prediction, and molecule taste prediction. FPU 102 then generates new preparation instructions that are similar to the food to be created. A profile of the food to be created may be generated from the subjective food tastings, analytical data (e.g., liquid chromatography mass spectrometry), and other information discussed herein.

As shown in FIG. 1, FPU 102 includes a molecular embedder 104 capable of performing a molecular embedding process. The molecular embedding process can produce a representation of the chemical and structural information of a mono-molecular tastant substance from which its flavor profile can be predicted. A tastant substance is any substance capable of producing a taste sensation (e.g., eliciting gustatory and/or olfactory excitation).

In some embodiments, molecular embedder 104 is implemented as a learned model that conceptually follows an auto-encoder architecture. The input to the encoder model is a molecular profile that includes the molecular structure and its chemical and physical properties, which is collectively denoted by the vector m. The output of the encoder model is a latent vector z=E(m). A decoder D is a learned model that receives a latent vector z representing the mono-molecular tastant substance and predicting a property of the mono-molecular tastant substance.

In some embodiments, multiple decoding heads are used, such as:

1. Dₐᵤₜₒ≈E⁻¹—A model predicting the molecular profile vector m itself. The model ensures that Dₐᵤₜₒ∘E≈Id makes the latent representation complete about the input molecule.

2. Dₛₑₙₛ—A model predicting the sensory response of certain gustative and olfactory receptor cells.

For simplicity of explanation, when describing the systems and methods herein, the explanation may refer to the encoder model as a deterministic one. A specific embodiment may instead represent, in some parametric form, the distribution of E(m) in the latent space.

As illustrated in FIG. 1, FPU 102 also includes a mixture modeler 106 capable of producing a representation of composite tastants that include multiple molecules. In some embodiments, mixture modeler 106 is a learned embedding model that receives an unordered collection Z={z₁, z₂, . . . , zₙ} of a fixed arbitrary number n of molecular embeddings. Mixture modeler 106 also receives a vector α=(α₁, α₂, . . . , αₘ) on the probability simplex representing their relative quantities in the mixture. Mixture modeler 106 produces an output that is another latent vector w=M(Z, α). For simplicity and based on a simple transitivity property, the following discussion assumes n=2, such that w=M(z₁, z₂, α, 1−α).

In some implementations, mixture modeler 106 is built to approximately satisfy homogeneity and additivity under the mixture, such as:

M(z₁,z₂,α,1−α)=αM(z₁)+(1−α)M(z₂)

In some embodiments, for purposes of convenience, the coordinate system is defined such that water is represented as zero.

In particular implementations, using mixture modeler 106 and asserting one of the mixands to be a solvent (e.g., water), the systems and methods can define another decoder head operating on the mixture representation space:

D_{subj}—A model predicting the subjective flavor profile. For example, in the case of a molecule m at concentration α in water, D_{subj}(αM∘E(m))=f predicts the perceived flavor characteristics, such as flavor categories, flavor feature scores, and relations to reference flavors, which are collectively denoted by the (pseudo-) vector f.

In some embodiments, the described systems and methods may assert the same space suiting both mono-molecular and mixture embeddings. In these implementations, the systems and methods use z and M(z) interchangeably (e.g., referring to both as z), such that the systems and methods may assume M∘E in place of E.

In some embodiments, FPU 102 further includes a preparation process modeler 108 capable of representing the effect of cooking and preparation processes on the latent representation. In some situations, a preparation process model may also be referred to as a precision graph or cooking graph.

In particular implementations, preparation process modeler 108 models a single step of the preparation process as a transformation of the latent space T(w)=w′. Using these terms, preparation instructions can be thought of as the composition of binary mixture and unary preparation operations. For example:

T₂(M(T₁(M(z₁,z₂,α)),z₃,α′)=T₂(α′T₁(αz₁+(1−α)z₂)+(1−α′)z₃).

In some embodiments, such a sequence can be represented as a tree with basic mono-molecular ingredients as the leaf nodes and the final food product at the root. The ingredients themselves, Z=(z₁, z₂, . . . , zₙ) and their relative quantities α=(α₁, α₂, . . . , αₙ) can be referred to as the formula of the food, which is different from the chemical formula. In some implementations, preparation instructions may be represented using a shorthand notation T(Z, α).

As shown in FIG. 1, FPU 102 also includes a virtual tasting system 110 capable of providing a virtual tasting room for testing new food products, food ingredients, and the like. In some embodiments, virtual tasting system 110 can predict which users may like a particular food product and which users may be the best testers of new food products or testing two or more similar food products.

As discussed herein, virtual tasting system 110 may support food testing and obtaining feedback on new food products using a smaller group of human testers. Instead of testing food products with a large number of random people, virtual tasting system 110 can provide valuable feedback on new food products using a smaller number of human testers. For example, the human testers for a new food product may be selected based on the human testers' food preferences, previous tasting event results, and the like.

In some embodiments, a tasting event produces various results that may include data related to taster preferences for one or more food products or compounds. Based on these results, each taster's profile may be updated based on their tasting preferences, and each food product's profile may be updated based on the tasting results from multiple tasters.

In some embodiments, virtual tasting system 110 may implement graph learning methods by, for example, predicting a taster's response to a substance. Based on sparse data collected from multiple tasters related to multiple substances, a deep neural network may be trained that recreates the geometry of the taster's space (e.g., the intra tasters relations) and the geometry of the substance space (e.g., the intra substance relations). Additionally, the deep neural network may be trained to recreate the interrelation between the tasters' graph and the substances' graph. In some embodiments, virtual tasting system 110 also supports the generation of new tasters, based on the required demographic and other background questionnaires, and prediction of the new tasters' response to a variety of substances.

In some embodiments, FPU 102 further includes a food model trainer 112 capable of training food models using a multi-task learning approach. In some implementations, individual models (e.g., molecular embedding models, mixture models, and preparation process models) can be pre-trained using individual sets of tasks followed by joint fine-tuning. Example learning tasks may include the following:

1. Homogeneity: asserting that given mixtures of ingredients z₁, z₂ in concentrations α, 1−α, and the flavor profile f of the mixture:

D_{subj}(αz₁+(1−α)z₂)=f

2. Transformed flavor profile: given pairs of flavor profiles (f, f′) of ingredients before and after a certain preparation process (e.g., heating to 180 degrees Celsius for 15 minutes), the transformed model can be trained by minimizing the discrepancy of the predicted taste profiles, D_{subj}∘T(f) and D_{subj}(f′).

3. Transformed chemistry: given pairs of chemical compositions ((Z, α), (Z′, α′)) of ingredients before and after a certain preparation process, the transformation model can be trained by minimizing the discrepancy of the predicted molecular profiles, T(α₁z₁+ . . . +αₙzₙ) and α′₁z′₁+ . . . +α′_{n′}z′_{n′}.

As shown in FIG. 1, FPU 102 also includes an inverse modeler 114, which takes the approach of designing a food product in an inverse manner. For example, inverse modeler 114 may solve an inverse problem that includes finding a formula having n molecular ingredients M=(m₁, m₂, . . . , mₙ), their quantities α, and the preparation process T(E(M), α). Inverse modeler 114 attempts to satisfy the following list of constraints. In particular embodiments, some or all of the constraints can be equivalently cast as optimization objectives.

1. Number of ingredients

2. Similarity to a target flavor profile D_{subj}∘T(E(M), α)=f_{target}, where f_{target} denotes the target flavor profile

3. Nutritional values of the molecular ingredients

4. Product cost including the sum of the cost of each mᵢ weighted by αᵢ and, in some situations, by the cost of all preparation stages comprising T.

The solution of the inverse problem can be carried out using regular backpropagation techniques.

In the case where the encoder E is stochastic, rather than getting a single solution, the systems and methods produce a posterior distribution from which multiple solution candidates can be sampled.

In some embodiments, FPU 102 further includes a preparation instruction manager 116 capable of storing and managing various preparation instructions. For example, preparation instruction manager 116 may track various ingredients, mixture ratios, and processing steps for different preparation instructions. Additionally, preparation instruction manager 116 may record tasting results (both subjective and objective) for various preparation instructions so the data can be used for creating different preparation instructions in the future. Preparation instruction manager 116 may also monitor and record visual, mechanical, and chemical properties of the prepared food.

In some embodiments, environment 100 further includes subjective flavor measurement data 118, objective flavor measurement data 120, ingredient data 122, and preparation instruction data 124. Subjective flavor measurement data 118 may include subjective results associated with an ingredient or preparation instruction by a human user. For example, subjective flavor measurement data 118 may include human user opinions regarding taste, texture, odor, and the like for a particular preparation instruction or ingredient.

In some embodiments, objective flavor measurement data 120 includes objective results associated with an ingredient or preparation instruction by a human user. For example, objective flavor measurement data 120 may include objective flavor profile data that is created or predicted using the systems and methods described herein. The objective flavor profile data may include predicted data regarding taste, texture, odor, and the like for a particular preparation instruction or ingredient.

Ingredient data 122 may include information associated with particular ingredients, such as an ingredient flavor profile, taste testing results associated with the ingredient, preparation instructions that include the ingredients, and the like. Preparation instruction data 124 may include information associated with various preparation instructions. In some embodiments, preparation instruction data 124 includes preparation instruction ingredients, preparation instruction mixing instructions, preparation instruction process, preparation instruction flavor profiles, preparation instruction taste testing results, and the like.

In some embodiments various ingredient data and preparation instruction data may be accessed or received from public databases combined with a measured outcome (e.g., objective or subjective features). In some implementations, the systems and methods described herein may perform pairwise comparisons or absolute taste grades with respect to different features, flavor keywords, and the like. In the case of absolute taste grades, the systems and methods may add heads that predict those characteristics.

It will be appreciated that the embodiment of FIG. 1 is given by way of example only. Other embodiments may include fewer or additional components without departing from the scope of the disclosure. Additionally, illustrated components may be combined or included within other components without limitation.

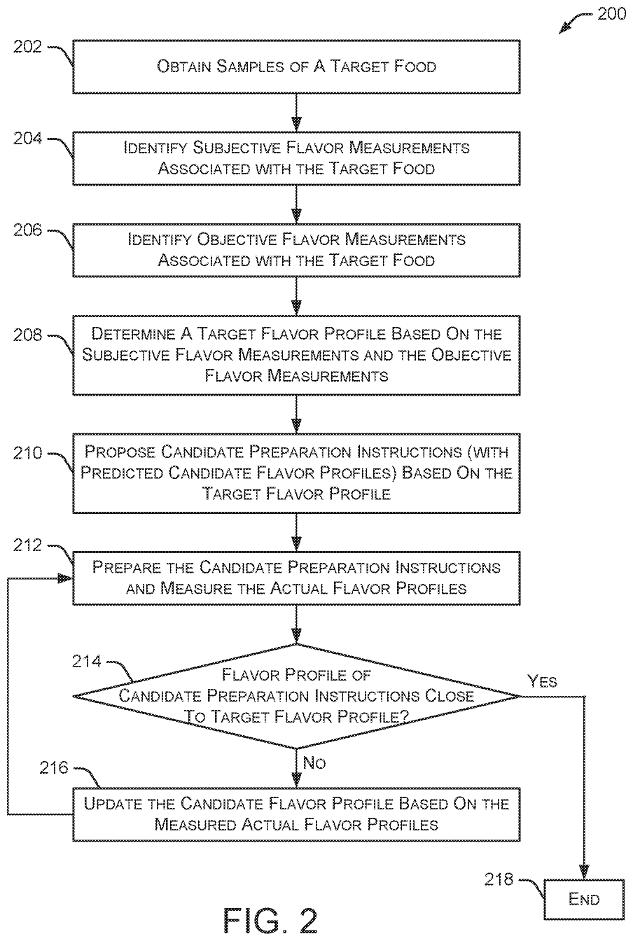

FIG. 2 is a flow diagram illustrating an embodiment of a process 200 for preparing and testing new preparation instructions. Initially, process 200 obtains 202 samples of a target food. In some embodiments, a target food may be a traditional food that is being copied by creating new preparation instructions with different ingredients, but a similar flavor profile. For example, the target food may be a traditional food that includes one or more animal-based ingredients. The systems and methods described herein are used to prepare a new version of the traditional food without animal-based ingredients, but maintaining the traditional food's flavor profile.

Process 200 continues by identifying 204 subjective flavor measurements associated with the target food. For example, the subjective flavor measurements may include taste, texture, smell, and the like. In particular implementations, the subjective flavor measurements are based on responses from human users who tasted the target food.

Process 200 then identifies 206 objective flavor measurements associated with the target food. For example, the objective flavor measurements may include physical and chemical information that may be used to predict taste, texture, smell, and the like. In some embodiments, the objective flavor measurements may be obtained as predictions from virtual tasting system 110 and other components of FPU 102.

The process continues by determining 208 a target flavor profile based on the subjective flavor measurements and the objective flavor measurements. This target flavor profile is used to create new preparation instructions with the same, or similar, flavor profiles as the existing food product. Process 200 then proposes 210 one or more candidate preparation instructions with predicted candidate flavor profiles based on the target flavor profile. In some embodiments, the candidate preparation instructions are expected to have predicted candidate profiles that are close to the target flavor profile.

The process continues by preparing 212 the one or more candidate preparation instructions and measuring the actual flavor profiles of the candidate preparation instructions. The process then compares the actual flavor profiles of the candidate preparation instructions to the target flavor profile. Process 200 continues by determining 214 whether the actual flavor profiles of the candidate preparation instructions are close to the target flavor profile. If the actual flavor profiles of the candidate preparation instructions are close to the target flavor profile, the process ends at 218. In some embodiments, the candidate preparation instructions that are close to the target flavor profile may be tested by one or more human users to determine whether the flavor of the food product created with one or more candidate preparation instructions is a viable replacement for the target food.

If the actual flavor profiles of the candidate preparation instructions are not close to the target flavor profile, process 200 updates 216 the candidate flavor profile based on the measured actual flavor profiles. The process then returns to 212, where the updated candidate preparation instructions are prepared and their actual flavor profiles are measured. The process further determines whether the actual flavor profiles of the updated candidate preparation instructions are close to the target flavor profile. This process of updating candidate preparation instructions and determining updated actual flavor profiles is repeated until the flavor profile of one or more candidate preparation instructions is close to the target flavor profile.

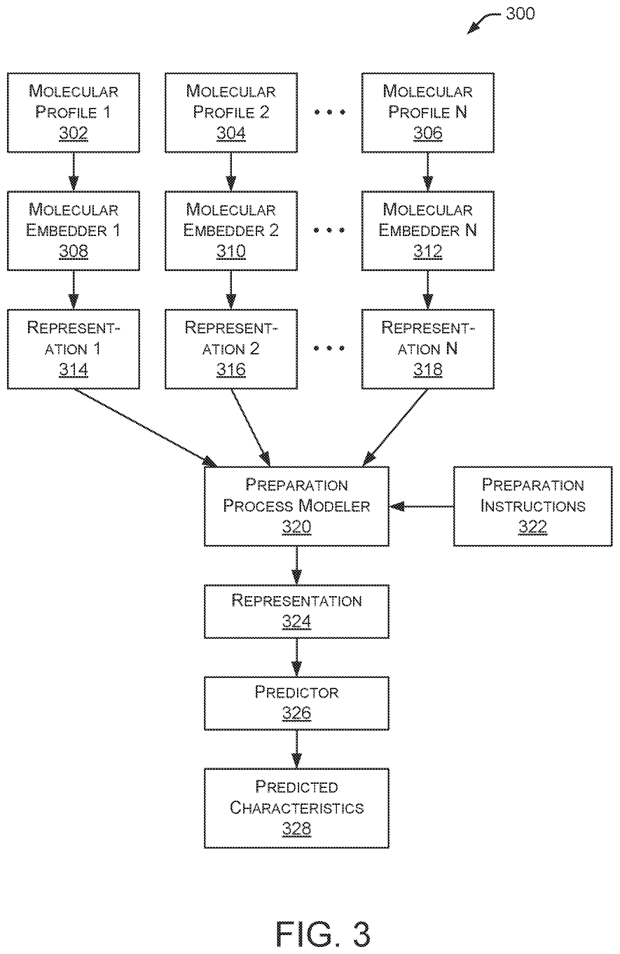

FIG. 3 is a block diagram illustrating an embodiment of a process flow 300 for predicting characteristics of a particular preparation instruction. As shown in FIG. 3, process flow 300 receives multiple molecular profiles 302, 304, and 306. Each molecular profile 302-306 defines various properties of a molecule or molecular structure that may be included in the results of a preparation instruction or other mixture. The molecular profiles 302-306 are provided to a molecular embedder 308, 310, 312, respectively. Molecular embedders 308-312 may be similar to molecular embedder 104 shown in FIG. 1 and discussed herein.

In process flow 300, each molecular embedder 308, 310, 312 generates a representation 314, 316, 318, respectively. Representations 314-318 of each molecule are vectors created via a (trainable) non-linear map of the input data. Each representation 314-318 contains enough dimensions such that the corresponding decoder heads can extract the required information with sufficient precision.

In some embodiments, the representations 314-318 are provided to a preparation process modeler 320. Preparation process modeler 320 may be similar to preparation process modeler 108 shown in FIG. 1 and discussed herein. Preparation process modeler 320 also receives preparation instructions 322, which may describe how the multiple molecular profiles 302-306 are mixed and processed.

Preparation process modeler 320 receives the representations of the input ingredients and generates a representation 324 of the prepared ingredient.

In some embodiments, representation 324 is provided to a predictor 326. Predictor 326 represents decoder heads that extract different objective and subjective characteristics from the representation vector regarding the food product being represented. In some embodiments, predictor 326 generates any number of predicted characteristics 328 related to the food product associated with representation 324. For example, predicted characteristics 328 may include a flavor profile associated with the food product identified in representation 324.

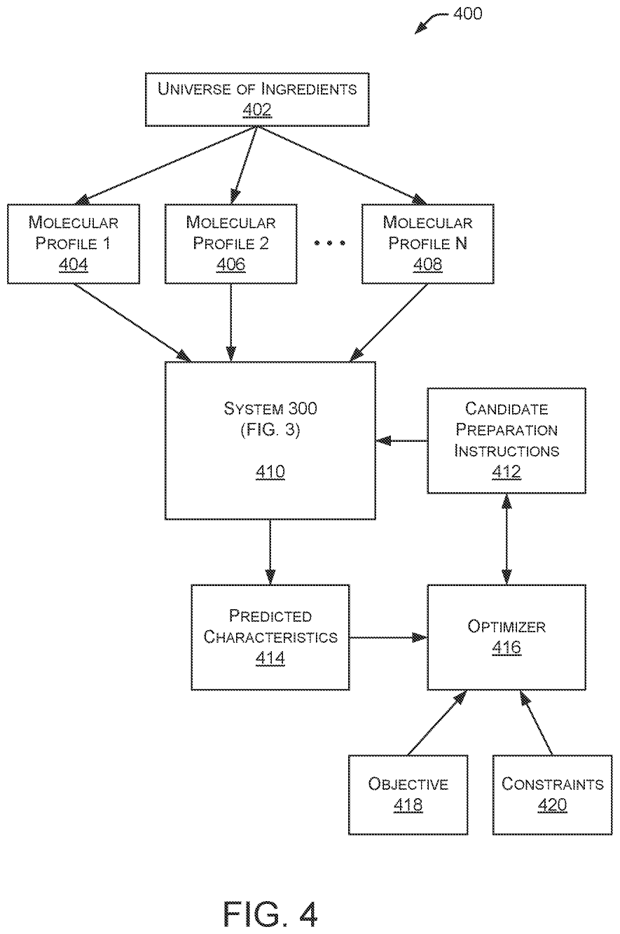

FIG. 4 is a block diagram illustrating an embodiment of a process flow 400 for optimizing creation of new preparation instructions. In the example of FIG. 4, one or more molecular profiles 404, 406, and 408 are selected from a universe of ingredients 402. Each molecular profile 404-408 defines various properties of a molecule or molecular structure that may be included in a preparation instruction or other mixture. The molecular profiles 404-408 are provided to a system 410 of the type shown in FIG. 3.

In some embodiments, system 410 receives a list of ingredients 404-408 and instructions about their preparation 412 (e.g., preparation instructions), then predicts a set of characteristics 414 of the final food product. Optimizer 416 may decide how to modify the candidate preparation instructions 412 to better match the objective or constraints. In some implementations, system 410 is the forward model that is inverted in the inverse modeler.

As shown in FIG. 4, system 410 generates candidate preparation instructions 412. System 410 also communicates predicted characteristics 414 to an optimizer 416. Optimizer 416 may also receive candidate preparation instructions 412. Optimizer 416 also receives objective information 418 and constraints information 420. In some embodiments, objective information 418 and constraints information 420 may be used by optimizer 416 to optimize a particular recipe. In particular implementations, optimizer 416 is part of FPU 102 working in the inverse mode (e.g., proposing new preparation instructions). For example, optimizer 416 may optimize the preparation instructions.

In the forward mode, given preparation instructions, the systems and methods described herein predict the characteristics of the preparation instructions. In the inverse mode, given particular target characteristics, the systems and methods find preparation instructions that satisfy the target characteristics.

In many situations, the subjective evaluation of food products traditionally involves tasting panels. A tasting panel is a group of appropriately trained experts that are given several food products to taste. At the end of the tasting panel, each taster completes a questionnaire comprising sensory (e.g., describing or comparing flavors) and hedonic (e.g., like or don't like) questions. In many cases, the process may be lengthy and costly due to the number of participants and the number of questions asked. The systems and methods described herein may alleviate the burden of the tasting panel's work.

As discussed herein, a list of evaluated food products may be designated as f₁, . . . , f_{K}, where each product fₖ is represented by a vector containing the product's profile. The discussion herein may further assume that there exists some kernel function κᵢⱼ^{F}=κ^{F}(fᵢ, fⱼ), which allows the systems and methods to determine the similarity of the food products, such that they can be organized into a weighed undirected graph [symbol] ^{F}, with the edge weights indicating the similarity of the food product.

Similarly, the discussion herein will denote by t₁, . . . , t_{M} the set of tasters with corresponding profiles (e.g., a taster's profile may include geographic, ethnic, and taste preference information), also arranged into a weighed graph [symbol] ^{T} whose edge weights κᵢⱼ^{T}=κ^{T}(tᵢ, tⱼ) represent the tasters' pairwise similarities.

Additionally, q₁, . . . , q_{N} denotes the set of questions, whose similarity is represented by the weighed graph [symbol] ^{Q} with the edge weights κᵢⱼ^{Q}=κ^{T}(qᵢ, qⱼ).

As discussed herein, the role of the tasting panel is to collect a third-order K×M×N tensor X whose entry xₖₘₙ represents the answer of taster m to question n about food product k. The answers may be either numerically or categorically valued. A particular questionnaire may contain both types of questions. In the discussion contained herein, for simplicity of exposition, the discussion assumes all the questions to be of the same type (e.g., numerical or categorical). However, from the teachings of this disclosure, the combination of both types will be clear to a person skilled in the art.

In some embodiments, the tensor X is redundant in the sense that the entirety of its entries is predictable from a subset thereof. This discussion denotes by C a subset of indices on which the tensor is measured (that is, if (k, m, n)∈C, it means that taster m was asked question n about food product k). The set C does not need to have a Cartesian product form, meaning that the same taster may be asked different subsets of questions about different subsets of food products. This discussion denotes by X|_{C} the subset of the tensor entries that were actually collected. The remaining entries are latent and have to be estimated using a computational tensor completion process.

Since there exist infinitely many ways to complete X given the inputs X|_{C}, the systems and methods may impose regularity of X with respect to the structure of the product graph [symbol] = [symbol] ^{F}□ [symbol] ^{T}□ [symbol] ^{Q} and algebraic regularity such as low tensor rank.

The tensor completion problem can be therefore expressed as the optimization problem of the form

$${min}_{\hat{X} ∈ {R}^{K ⨯ M ⨯ N}} ℓ ( \hat{X} {\vert}_{C} , X {\vert}_{C} ) + ρ ( \hat{X} ) + {ρ}_{alg} ( \hat{X} ) ,$$

where \hat{X} denotes the estimated full tensor, [symbol] is a loss function measuring the discrepancy between the actual measurements X|_{C} and the corresponding subset of the estimated tensor, \dot{X}|_{C}, [symbol] is a penalty function promoting geometric regularity with respect to the product graph [symbol] on which \hat{X} lives, and ρ_{alg} is a penalty function promoting algebraic regularity of the solution.

The choice of the loss function depends on the meaning of the values of the answers to the questions contained in the tensor. In the case of numerical values, the most obvious choices are the [symbol] ₂ distance (regression of the mean), the [symbol] ₁ distance (regression of the median) and, more generally, the pinball loss (regression of a specific quantile level). In the case of categorical values, the typical choice is a cross-entropy loss. While these specific choices are given as examples of the loss function, their combination or other conceptually similar loss functions will be understood to a person skilled in the art.

In some embodiments, the assumption of band-limitedness (a.k.a., smoothness) of the signal on the product graph [symbol] may be appropriate. This assumption may be embodied by expressing a geometric regularity term in the form of the Dirichlet energy on [symbol] ,

[symbol]

(X)=(X, [symbol] X)

where Δ [symbol] is the Laplacian of [symbol] and <⋅, ⋅> is an inner product on [symbol] .

In the case of a separable product graph, the latter energy can be split into dimension-wise energies

$${ρ}_{} ( X ) = \text{?} ( \text{?} {X}_{, m , n} ) + \text{?} ( \text{?} ) + \text{?} ( \text{?} ) .$$

$$\text{?} \text{indicates text missing or illegible when filed}$$

In some embodiments, an algebraic regularity term promotes low-rank structure of the estimated tensor. Since rank by itself is intractable ρ_{alg}(X) may contain the sum of the singular values in the tensor singular value decomposition (SVD) of X or the sum of the singular values in matrix slices of the tensor,

$${ρ}_{alg} ( X ) = {\sum }_{in} {σ}_{i} ( \text{?} ) + {\sum }_{im} {σ}_{i} ( \text{?} ) + {\sum }_{ik} {σ}_{i} ( \text{?} ) .$$

$$\text{?} \text{indicates text missing or illegible when filed}$$

where σᵢ indicate the singular values.

In some embodiments, in order to avoid the complexity of SVD, the solution \hat{X} can be factorized into low-rank factors.

In particular embodiments, an implicit rank regularization may be used by employing an optimization algorithm promoting low-rank solutions (e.g., stochastic gradient descent).

In some embodiments, a “cold start” is used in a situation when one or several new food products, tasters, or questions are added, and the answers need to be predicted for the extended tensor.

Without loss of generality, assume that a new food product f_{K+1} is added. Using its profile, the systems and methods described herein can create a new graph [symbol] ^{F} containing the previous K-node graph with the addition of a new node connected to the previous nodes with edges whose weights are given by κ(fₖ, f_{K+1}). The described optimization procedure can readily estimate the entries of κ(fₖ, f_{K+1}).

A similar procedure can be applied when multiple new food products or new tasters and/or questions are added.

In some embodiments, when no reliable way exists to estimate the similarity of food products, tasters, or questions, the kernels constructing the product graph metrics can be parametrized. In some implementations, the parameters are added to the optimization problem. Denoting by [symbol] (θ) a graph with weights stemming from a parametric kernel κ_{θ}, the optimization problem becomes

$${min}_{\hat{X} , θ} ℓ ( \hat{X} {\vert}_{C} , X {\vert}_{C} ) + {ρ}_{{θ}_{}} ( \hat{X} ) + {ρ}_{alg} ( \hat{X} ) .$$

The kernel κ_{θ} may have a separable form

κ_{θ}=κ_{θ}_{_{F}}⊗κ_{θ}_{_{T}}⊗κ_{θ}_{_{Q}}

given by the tensor product of kernels on

[symbol] _{F}, [symbol] _{T} and [symbol] _{Q}, and θ=(θ_{F}, θ_{T}, θ_{Q}).

In other embodiments, an inseparable kernel is employed.

In some embodiments, rather than collecting all the answers in a single batch, the described systems and methods may first collect X|_{C}_{₁} on a small set C₁, and produce the first estimate of the full tensor by solving

$${\hat{X}}_{1} = arg {min}_{\hat{X}} ℓ ( \hat{X} {\vert}_{{C}_{1}} , X {\vert}_{{C}_{1}} ) + {ρ}_{} ( \hat{X} ) + {ρ}_{alg} ( \hat{X} ) .$$

A set predictor then accepts the values of \hat{X}₁ to produce a new set C₂ disjoint with C₁, and new measurements X|_{C}_{₂} are collected on C₂. The optimization procedure is then applied again with the full input to produce a refined estimate of the full tensor

$${\hat{X}}_{2} = arg {min}_{\hat{X}} ℓ ( \hat{X} {\vert}_{{C}_{1} ⊔ {C}_{2}} , X {\vert}_{{C}_{1} ⊔ {C}_{2}} ) + {ρ}_{} ( \hat{X} ) + {ρ}_{alg} ( \hat{X} ) .$$

The process can be continued iteratively until a prescribed out-of-sample estimation error or combination of other stopping criteria is reached.

In some situations, human tasters are unable to reliably provide an absolute quantitative answer to tasting questions. For example, different people, or even the same person in different trials, may score the “sweetness” of a food product with a different number. A more reliable way of asking the same question is by making it relative. For example, asking which, out of a pair of food products, is sweeter.

When treating each question separately, the measurements consist of a set of binary or trinary values x_{k,k′, m}∈{−1, 0, +1} indexed by tuples of the form (k, k′, m) where (k, k′) is a pair of food products compared by taster m with respect to a specific feature in question (e.g., sweetness). The systems and methods may assume that the value +1 means that k>-k′ in the sense of that feature, −1 means that k-<k′ in the same sense, and 0 means that k′˜′k′.

Given such an input, the goal of the systems and methods described herein is to assign each food product k a numerical score yₖ, whose role is to order the food products according to the feature in question (e.g., ascending from plain to sweet). Furthermore, in some embodiments, the score can also reflect a distance between two food products in the sense of the measured feature. For example, a sequence of scores 1, 2, 3 and 1, 1.1, 10 has the same ordering, but in the latter case the first two food products are very similar in the sense of the feature, while the third food product has a much stronger difference.

In some embodiments, the input data can be represented as a weighted directed graph with the food products acting as the nodes, and the weight of each edge k→k′ determined by averaging (or otherwise aggregating) x_{k,k′,m} all over m. For example, if the tasters prevalently determined k sweeter than k′(x_{k,k′,m}=+1 for most m's), the edge k→k′ will have a positive weight close to 1, and k′→k will be equally weighted with an opposite sign.

In some implementations, a first step is to filter the graph for directed cycles as they clearly represent inconsistent data. Cycles of all lengths are enumerated and sorted by the weights of their constituent edges in ascending orders. A minimal number of smallest-weight cycles is removed until the graph becomes acyclic (a DAG).

A topological sorting algorithm is then applied to the preprocessed graph, assigning the vertices ordinal ranks yₖ∈{1, 2, . . . , K}, with lower yₖ's implying that k-<k′ for most k′, and higher yₖ's implying k>-k′ for most k′.

Topological sorting does not allow to measure meaningful distances between the produced scores yₖ. A more geometric algorithm may try to treat the yₖ's as embedding coordinates, minimizing a multidimensional scaling-like stress function

$$L ( y ) = {\sum }_{k , {k}^{′}} {\sum }_{m} d ( {y}_{k} - {y}_{{k}^{′}} , {x}_{k , {k}^{′} , m} )$$

where y=(y₁, . . . , yₖ), and d is some discrepancy measure (e.g., an [symbol] ₂ distance). Other conceptually similar loss functions can be used in other embodiments.

In some embodiments, a probabilistic variant of the above algorithm consists of treating the values {x_{k,k′,m}} for each pair (k, k′) as independent samples of a random variable and fit distribution parameters for each food product k explaining these measurements. Assuming, as an example of a normal model, each food product is assigned a scale parameter σₖ² in addition to the location parameter yₖ, and the following negative log likelihood function is minimized during filtering:

$$L ( y ) = {\sum }_{k , {k}^{′}} {\sum }_{m} ℓ ( {x}_{k , {k}^{′} , m} \vert {y}_{k} - {y}_{{k}^{′} ,} {σ}_{k}^{2} + {σ}_{{k}^{′}}^{2} ) ,$$

where [symbol] (⋅|y,σ²) is the negative log likelihood function of a Gaussian distribution with mean y and variance σ².

The described systems and methods support food testing and obtaining feedback on new food products using a smaller group of human testers. Instead of testing food products with a large number of random people, the systems and methods described herein can provide valuable feedback on new food products using a smaller number of human testers. For example, the human testers for a new food product may be selected based on the human testers' food preferences, previous tasting event results, and the like.

In some embodiments, a tasting event produces various results that may include data related to taster preferences for one or more food products or compounds. Based on these results, each taster's profile may be updated based on their tasting preferences, and each food product's profile may be updated based on the tasting results from multiple tasters.

In some embodiments, the systems and methods described herein implement graph learning methods by, for example, constructing an emulator that can predict a taster's response to a substance. Based on sparse data collected from multiple tasters related to multiple substances, a deep network may be trained that recreates the geometry of the taster's space (e.g., the intra tasters relations) and the geometry of the substance space (e.g., the intra substance relations). Additionally, the deep network may be trained to recreate the interrelation between the tasters' graph and the substances' graph. The described systems and methods also support the generation of new tasters, based on the required demographic and other background questionnaires, and prediction of the new tasters' response to a variety of substances.

Graph neural networks are types of neural network based models where the input layers, intermediate layers, and/or the output layers handle mathematical graphs. For example, the graphs may include a collection of vertices and edges (V, E). The main property of graph processing is the invariance or equivariance to permutation on the set of vertices (also referred to as nodes). In some embodiments, graph neural networks capture the dependence of graphs and are able to learn the relational information among graph elements. Therefore, these models are used to classify nodes and/or edges in a specific graph (e.g., predict certain attributes related to nodes or edges) as well as to predict functionally of whole graphs.

The systems and methods described herein include graph neural networks that are trained to predict the full response set of tasters to a panel of edible compounds based on sparse sampling of a subset of tasters and a subset of samples (e.g., food compounds). A compound may be a food item, a food ingredient, a spice, a flavoring, and the like. In some embodiments, the underlying graph represents high dimensional data of a high number of unknown features both of the tasters and the samples. In some situations, only a small subset of features is known such that nodes and edges are related to tasters and some partial information regarding their preferences. The model learns to complete the unknown features and reveals the interrelation between tasters and compounds.

In some embodiments, the network further reconstructs the latent graph of intra-relation among the tasters. That is, a graph where each node is a taster and the weight assigned to each edge is proportional to the similarity between the tasters. Similarity between tasters is the property which correlates to the probability that two tasters will give the same score or assign the same taste to all samples. This provides a way of determining the key properties among tasters that largely impact their organoleptic response and scoring. When a new node is added to the graph (e.g., a new taster is introduced), firstly, a “cold start” estimation localizes the new node in the graph based on the demographic and basic questionnaire answers. This provides a rough estimate of the new taster's organoleptic preference. This can be further fine-tuned as further questions are answered by the tasters until the confidence score for the edges' weights between the taster to all other nodes is high enough, fully localizing the new node.

Similarly, a latent graph of intra-relation among the compounds is generated. In some embodiments, this latent graph of intra-relation among the compounds is a graph where each node is a compound and the weight assigned to each edge corresponds to the similarity between these compounds in terms of physical and organoleptic attributes. This enables the clustering of compounds by their function and properties and also to establish key attributes that broadly influence tasters and their scoring by tasters. The same “cold start” estimation may be used here as well when a new node is added to the graph (e.g., a new compound is introduced) where the initial information used regarding this new node is their food group and known related values (e.g., nutritional values).

In some embodiments, the developed models can work as an offline tool to enrich existing surveys, meaning to improve surveys that have already been conducted in the past and provide additional insights, as well as an active recommendation tool which guides active surveys, meaning direct and advise in real-time which food to give to which tasters in the next tasting session as well as which questions are more relevant or indicative based on their scores in the previous session.

In some embodiments, the system is composed of two main blocks: (1) data preparation and preprocessing, and (2) graph neural network and/or a deep neural network based model. In particular implementations, there are two modes of operation: training mode and prediction mode. Example systems and methods to implement these two main blocks are described herein.

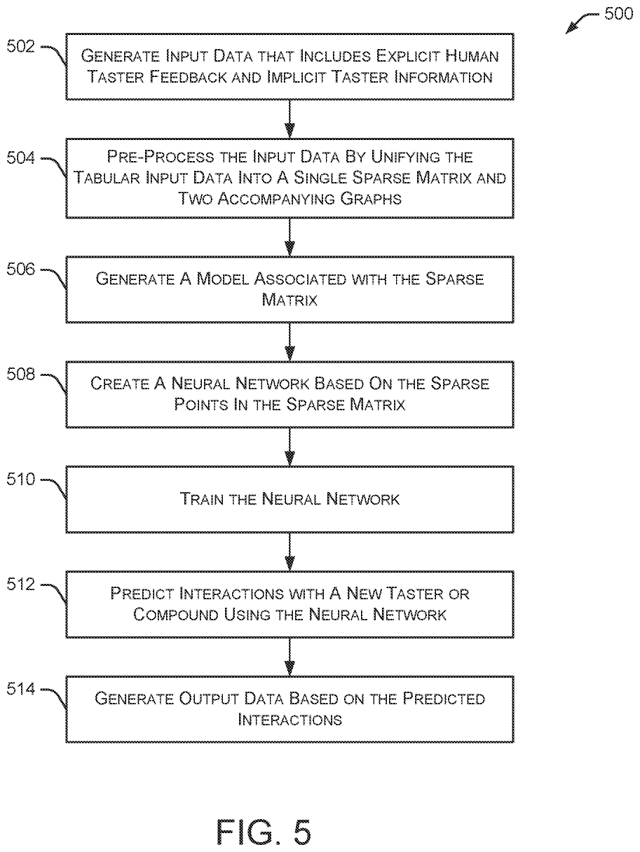

FIG. 5 is a flow diagram illustrating an embodiment of a process 500 for implementing a virtual tasting room and similar environments. In some embodiments, process 500 is implemented by a computing system such as a desktop computer, a server, or any other local or remote computing device.

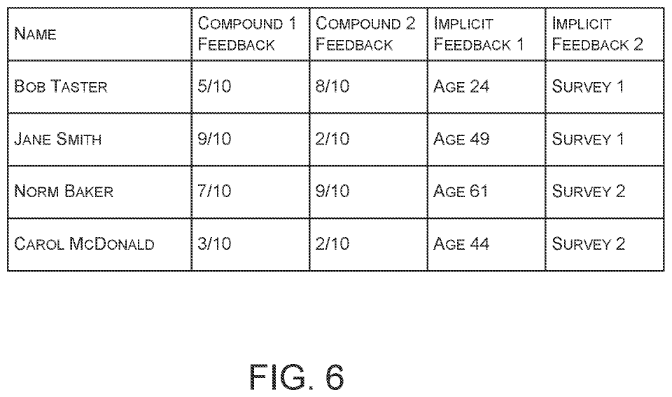

Initially, process 500 generates 502 input data that includes explicit human taster feedback and implicit taster information. In some embodiments, this input data may include tabular data such that there are multiple tables, where each table is structured into multiple rows and columns. For example, each row may contain information related to a particular taster. The columns represent various features that may be divided into two groups: 1) explicit taster feedback (e.g., taster feedback of a compound), and 2) implicit information (or side information) of tasters, compounds, and survey details. This implicit information may include, for example, taster characterization (e.g., demographics like age and gender, eating habits), survey specific details (e.g., date, goal), food compound attributes (e.g., type of product), and response/opinion to some features related to a food compound (e.g., texture, taste), or compounds (e.g., A better than B). The multiple columns may have mixed data types meaning they may contain different data types such as numbers, words, true/false values and they may relate to either the taster or the compound. FIG. 6 illustrates an example embodiment of a table of the type discussed above. Particular embodiments of the table shown in FIG. 2 may include any number of rows and any number of columns.

Referring to FIG. 5, process 500 may preprocess 504 the input data by unifying the tabular input data into a single sparse matrix and two accompanying graphs. In some embodiments, the sparse matrix depicts the relationship between tasters and compounds. It is the adjacency matrix that shows the tasters' responses to compounds (e.g., one is represented as the rows and one as the columns). This matrix is sparse since only partial explicit feedback is available and the rest is unknown (since not all tasters rated all the compounds). The described systems and methods may construct two additional graphs, one corresponding with the rows of the graph and one with the columns (e.g., a taster graph and a compound graph). In some implementations, each graph encapsulates the taster-taster and compound-compound interrelations. The edges in these graphs represent the distance/similarity between tasters and between compounds, and are initiated using the side information (e.g., for the tasters, the demographics information is used and for the compounds nutritional values and/or ingredients).

Process 500 in FIG. 5 continues by generating 506 a model associated with the sparse matrix. In some embodiments, the model is mainly aimed at matrix completion, meaning that once trained it is able to predict the missing values in the sparse matrix (i.e., adjacency matrix). For example, the model may estimate for each taster, his/her response to a given compound. The model solves this task by learning the decomposition of the sparse matrix into two matrices, one related to the user and one related to the compounds. In some implementations, the two matrices essentially embed tasters and compounds into low dimensional feature spaces that express the relationships between tasters (taster-taster) and between compounds (compound-compound) by learning a representation in which elements which resemble each other are closer to one another in the space.

As shown in FIG. 5, process 500 creates 508 a neural network based on the sparse points in the sparse matrix. In some embodiments, the neural network, such as a deep linear network or a graph based network, is designed to find the optimal decomposition of the observable sparse points such that the error of recovering the observed points from the prediction is minimized.

Process 500 further trains 510 the neural network using, for example, an optimal decomposition that is a decomposition that minimizes the rank of the sparse matrix. Since the model is based on a deep linear network, the rank constraint may be captured by the implicit regularization induced by gradient descent.

Process 500 continues by predicting 512 interactions with a new taster or compound using the neural network. In some embodiments, a cold start is when a new sample (taster or compound) is introduced with no previous tasting experience or tasting preference data. In the example of the cold start, the available features are used for “cold-start” by placing the sample in its respective graph, and the prediction of its interactions is initialized based on that. If there are some known interactions, applying the model on the newly observed sparse vector gives the full interaction vector.

Finally, process 500 generates 514 output data based on the predicted interactions. For example, the model may yield three types of outcomes: (1) rating prediction, (2) taster-taster interrelation, and (3) compound-compound interrelation.

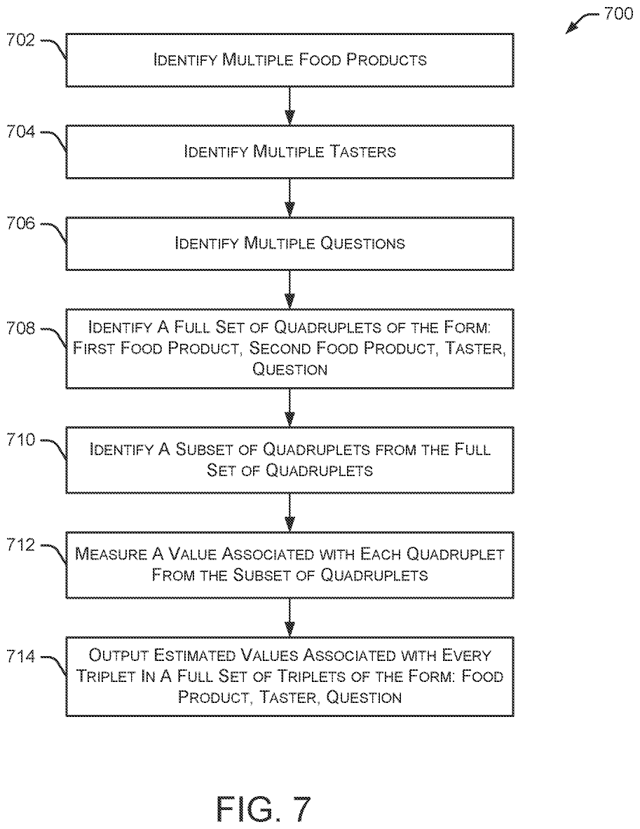

FIG. 7 is a flow diagram illustrating an embodiment of a process 700 for implementing a virtual tasting system. Initially, process 700 identifies 702 multiple food products and identifies 704 multiple tasters. Additionally, the process identifies 706 multiple questions, such as questions to ask the tasters about one or more food products.

Process 700 continues by identifying 708 a full set of quadruplets of the form: first food product, second food product, taster, question. In some embodiments, food products are given random but consistent labels and are served in identical conditions so the tasters do not know the product identity. Additionally, the order of the serving may be randomized to avoid biases. When analyzing the results of a tasting activity, the data are first analyzed and the results are shown with the arbitrary labels that are later converted to the real product names.

The process continues by identifying 710 a subset of quadruplets from the full set of quadruplets. In some embodiments, the subset of quadruplets can be selected randomly or selected systematically. In particular implementations, the subset of quadruplets may be refined based on an outcome of matrix completion on the previous coarser subset.

Process 700 measures 712 a value associated with each quadruplet from the subset of quadruplets. The process continues by outputting 714 estimated values associated with every triplet in a full set of triplets of the form: food product, taster, question. Quadruplets may include pairs of products (e.g., the input for pairwise comparison and pairwise ranking). Triplets are a particular case of quadruplets in which the products in the pair are identical. The latter case is the input to the matrix completion algorithm described herein.

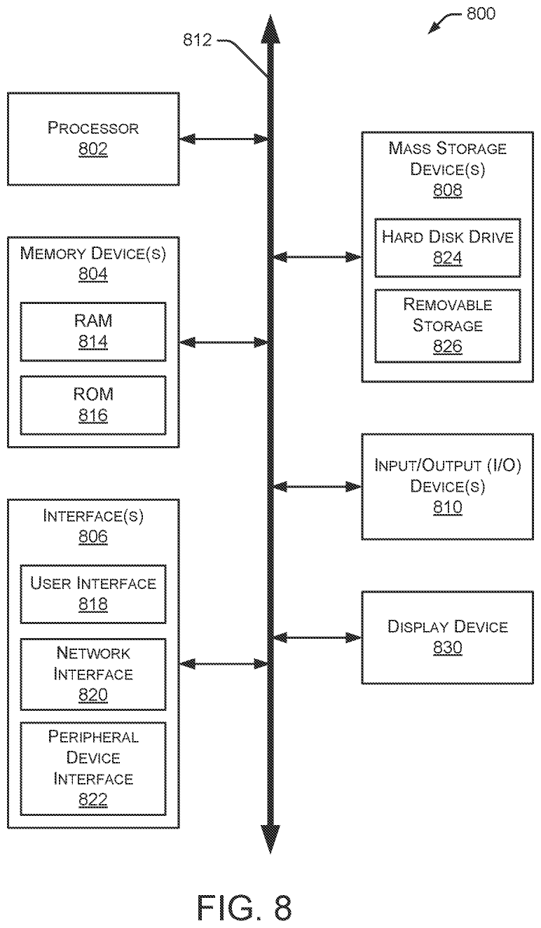

FIG. 8 illustrates an example block diagram of a computing device 800 suitable for implementing the systems and methods described herein. In some embodiments, a cluster of computing devices interconnected by a communication network may be used to implement any one or more components of the systems discussed herein.

Computing device 800 may be used to perform various procedures, such as those discussed herein. Computing device 800 can function as a server, a client, or any other computing entity. Computing device can perform various functions as discussed herein, and can execute one or more application programs, such as the application programs described herein. Computing device 800 can be any of a wide variety of computing devices, such as a desktop computer, a notebook computer, a server computer, a handheld computer, tablet computer and the like.

Computing device 800 includes one or more processor(s) 802, one or more memory device(s) 804, one or more interface(s) 806, one or more mass storage device(s) 808, one or more Input/Output (I/O) device(s) 810, and a display device 830 all of which are coupled to a bus 812. Processor(s) 802 include one or more processors or controllers that execute instructions stored in memory device(s) 804 and/or mass storage device(s) 808. Processor(s) 802 may also include various types of computer-readable media, such as cache memory.

Memory device(s) 804 include various computer-readable media, such as volatile memory (e.g., random access memory (RAM) 814) and/or nonvolatile memory (e.g., read-only memory (ROM) 816). Memory device(s) 804 may also include rewritable ROM, such as Flash memory.

Mass storage device(s) 808 include various computer readable media, such as magnetic tapes, magnetic disks, optical disks, solid-state memory (e.g., Flash memory), and so forth. As shown in FIG. 8, a particular mass storage device is a hard disk drive 824. Various drives may also be included in mass storage device(s) 808 to enable reading from and/or writing to the various computer readable media. Mass storage device(s) 808 include removable media 826 and/or non-removable media.

I/O device(s) 810 include various devices that allow data and/or other information to be input to or retrieved from computing device 800. Example I/O device(s) 810 include cursor control devices, keyboards, keypads, microphones, monitors or other display devices, speakers, printers, network interface cards, modems, lenses, CCDs or other image capture devices, and the like.

Display device 830 includes any type of device capable of displaying information to one or more users of computing device 800. Examples of display device 830 include a monitor, display terminal, video projection device, and the like.

Interface(s) 806 include various interfaces that allow computing device 800 to interact with other systems, devices, or computing environments. Example interface(s) 806 include any number of different network interfaces 820, such as interfaces to local area networks (LANs), wide area networks (WANs), wireless networks, and the Internet. Other interface(s) include user interface 818 and peripheral device interface 822. The interface(s) 806 may also include one or more user interface elements 818. The interface(s) 806 may also include one or more peripheral interfaces such as interfaces for printers, pointing devices (mice, track pad, etc.), keyboards, and the like.

Bus 812 allows processor(s) 802, memory device(s) 804, interface(s) 806, mass storage device(s) 808, and I/O device(s) 810 to communicate with one another, as well as other devices or components coupled to bus 812. Bus 812 represents one or more of several types of bus structures, such as a system bus, PCI bus, IEEE 1394 bus, USB bus, and so forth.

For purposes of illustration, programs and other executable program components are shown herein as discrete blocks, although it is understood that such programs and components may reside at various times in different storage components of computing device 800, and are executed by processor(s) 802. Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

While various embodiments of the present disclosure are described herein, it should be understood that they are presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the described exemplary embodiments, but should be defined only in accordance with the following claims and their equivalents. The description herein is presented for the purposes of illustration and description. It is not intended to be exhaustive or to limit the disclosure to the precise form disclosed. Many modifications and variations are possible in light of the disclosed teaching. Further, it should be noted that any or all of the alternate implementations discussed herein may be used in any combination desired to form additional hybrid implementations of the disclosure.

1. A method comprising:
identifying a plurality of food products;
identifying a plurality of tasters;
identifying a plurality of questions;
identifying a full set of quadruplets of the form: first food product, second food product, taster, question;
identifying a subset of quadruplets from the full set of quadruplets;
measuring a value associated with each quadruplet from the subset of quadruplets; and
outputting estimated values associated with every triplet in a full set of triplets of the form: food product, taster, question.

2. The method of claim 1, wherein the second food product is identical to the first food product.

3. The method of claim 1, further comprising identifying a similarity function associated with the plurality of food products.

4. The method of claim 1, further comprising identifying a similarity function associated with the plurality of tasters.

5. The method of claim 1, further comprising identifying a similarity function associated with the plurality of questions.

6. The method of claim 1, wherein the plurality of food products include single ingredients or multiple ingredients.

7. The method of claim 1, wherein the plurality of tasters include a plurality of individual people tasting the plurality of food products.

8. The method of claim 1, wherein the plurality of questions include topics such as savor, smell, texture, or mouthfeel.

9. A method comprising:
identifying a plurality of food products;
identifying a plurality of tasters;
identifying a plurality of questions;
identifying a full set of quadruplets of the form: first food product, second food product, taster, question;
identifying a subset of quadruplets from the full set of quadruplets;
measuring a value associated with each quadruplets from the subset of quadruplets;
identifying a plurality of at least one of new food products, new tasters, or new questions;
identifying an extended set of quadruplets of the form: first food product, second food product, taster, question, wherein the extended set of quadruplets includes the new food products, new tasters and new questions; and
outputting estimated values associated with every quadruplet from the extended set of quadruplets of the form: food product, taster, question.

10. The method of claim 9, further comprising identifying a similarity function associated with the plurality of food products.

11. The method of claim 9, further comprising identifying a similarity function associated with the plurality of tasters.

12. The method of claim 9, further comprising identifying a similarity function associated with the plurality of questions.

13. The method of claim 9, wherein the first food product is identical to the second food product.

14. A method comprising:
identifying a plurality of food products;
identifying a plurality of tasters;
identifying a plurality of questions;
identifying a full set of triplets of the form: food product, taster, question;
identifying a subset of triplets from the full set of triplets;
measuring a value associated with each triplet from the subset of triplets;
estimating a graph associated with the similarities of each of the pluralities of food products, tasters and questions; and
outputting estimated values associated with every triplet in the full set of triplets.

15. The method of claim 14, further comprising identifying a parametric family of similarity functions.

16. The method of claim 14, further comprising estimating the parameters of the family of similarly functions from which the graph is estimated.

17. A method comprising:
identifying a plurality of food products;
identifying a plurality of tasters;
identifying a plurality of questions;
identifying a full set of quadruplets of the form: first food product, second food product, taster, question;
identifying an initial subset of quadruplets from the full set of quadruplets;
measuring a value associated with each quadruplets from the initial subset of quadruplets;
outputting estimated values associated with every quadruplet in the full set of quadruplets of the form: food product, taster, question;
identifying a refined subset of quadruplets from the full set of quadruplets;
measuring a value associated with each quadruplet from the refined subset of quadruplets; and
iteratively refining the subset of quadruplets.

18. The method of claim 17, further comprising identifying a similarity function associated with the plurality of food products.

19. The method of claim 17, further comprising identifying a similarity function associated with the plurality of tasters.

20. The method of claim 17, further comprising identifying a similarity function associated with the plurality of questions.
